(19) United States
(12) Patent Application Publication
(10) Pub. No.: US 2023/0220155 A1
(43) Pub. Date: Jul. 13, 2023

(54) AQUEOUS COATINGS MADE FROM POLYHYDROXYALKANOATE (PHA) CAKE

(71) Applicant: Meredian, Inc., Bainbridge, GA (US)

(72) Inventors: Joe B. Grubbs, III, Bishop, GA (US); Michael J. Joyce, Athens, GA (US); Karson Durie, Athens, GA (US)

(73) Assignee: Meredian, Inc., Bainbridge, GA (US)

(21) Appl. No.: 18/153,729

(22) Filed: Jan. 12, 2023

Related U.S. Application Data
(60) Provisional application No. 63/325,318, filed on Mar. 30, 2022.
(60) Provisional application No. 63/266,702, filed on Jan. 12, 2022.

(51) Int. Cl. C08G 63/06; C12P 7/625; D21H 19/28

(52) U.S. Cl. CPC C08G 63/06; C12P 7/625; D21H 19/28; C08G 2230/00; C08G 2150/00

(57) ABSTRACT

A biodegradable aqueous mixture for coating substrates is disclosed, which includes from about 35 to about 75 weight percent water and from about 25 to about 65 weight percent solids. The solids in turn are made up of from about 40 to about 99 weight percent polyhydroxyalkanoates based on the total dry weight of the solids. Moreover, the polyhydroxyalkanoates are in the form of polyhydroxyalkanoate particles having a moisture content of no less than about 1% by weight prior to mixing with the water and a Dv (90) particle size of no more than about 10 microns, as determined using ISO 8130-13:2019.

### FIELD

This invention relates to the field of polyhydroxyalkanoate (PHA) production. More particularly, this invention relates to the production of a novel form of PHA, referred to as PHA cake herein, and to a coating incorporating this PHA cake.

### BACKGROUND

Polyhydroxyalkanoate (PHA) is typically produced by the fermentation of biomass of bacteria or other microorganisms in a bioreactor. First, PHA is synthesized by and accumulated in the cells of the microorganisms. Then, after the PHA is grown in biomass, there is a multi-step process to lyse the bacteria and separate the PHA from the cell debris. Finally, the biomass is dried, such as in an oven, and processed into a dry powder.

The dry powder can be stored and shipped but must be re-wet before use in suspension, emulsion, dispersion, or colloidal forms. Re-wetting typically requires sonication, ultrasonication, low or high shear, and other processes to break the dried particles down into a smaller particle size, and to enable surfactants to wet-out particles. These processes used to re-wet the material take substantial time and energy, and can result in significant foaming. Like all powders, dried PHA includes hazards such as explosion and inhalation. Further, later hydration or other solvation of the dried powder may increase the level of additives such as dispersing and wetting agents (surfactants) to get the particles to disperse and wet-out. These levels of additives tend to decrease the regulatory acceptance, biodegradability, and compostability of the material. The life-cycle assessment of PHA formed into powder is diminished due to the required increased energy and water consumption resulting from the use of drying ovens to create PHA powder, and additional water for re-wetting processes. This overall, reduces the sustainability of PHA powder materials.

Therefore, what is needed is a method of producing PHA that tends to reduce issues such as those introduced above, at least in part.

It would also be desirable to provide a biodegradable aqueous mixture for coating substrates which comprises PHA produced so produced.

### SUMMARY

The above and other needs are met by a method of forming polyhydroxyalkanoate (PHA) that eliminates heated drying processes, need for re-wetting, increased use of surfactants for dispersing, increased energy usage for high shear dispersing/sonication, significant particle agglomeration resulting in increased particle size as compared to that of those resulting from post lysis. The resulting PHA is newly referred to as PHA cake. The term “cake” is new in this disclosure and has not heretofore been used in the PHA processing industry to describe an end-use material having the properties described above.

PHA cake made according to the methods described here can be either water or solvent-based and is well-suited for formulation of dispersions, colloids, suspensions, coatings, and similar materials. Further, this material is well suited to be used as is into processes which utilize organic and inorganic solvent systems. Examples of formulations made using this cake material include those such as barrier coatings and surface coatings. Further examples for the use of this material include the inclusion or embedding of the electrolytes or material into or onto paper products, dispersions, colloids, emulsions, films, and heat seals. The PHA product is a dispersion of PHA in a liquid, such as water or ethanol, but acts much like an emulsion.

The PHA is created using steps similar to a normal biomass process, but after lysis and protein extraction, the PHA is washed back and forth in an alternating manner with organic and inorganic solvents, such as an alcohol and water, with pressing, terminal filtration, cross flow filtration, decanting, or combination of the two between the washing steps. Blowing air or nitrogen preferably at room temperature can also be performed to yield a PHA cake with no less than about 5% liquid by weight, and in some embodiments, about 60 (+/−10) % PHA and 40 (+/−10) % liquid, by weight.

The final PHA cake product can then be used in a variety of different ways, such as being placed in a mixer to which water, surfactants, preservatives, rheology modifiers, thickening agents, wetting agents, defoamers, biocides, fillers, binders, and dispersing agents are added. These products can then be used in a variety of different ways, as described elsewhere herein.

By not undergoing a heated drying process with elevated temperatures, the PHA cake retains a smaller PHA particle size, when compared with a standard PHA product that has been baked dry to a solid. The smaller particle size further enables better wetting of the PHA particles, reduction in the time/energy for dispersing of particles, reduction in the use of additives (i.e., surfactants) to prevent flocculation and particle agglomerate, and ultimately increases processability, efficiency, and the sustainability of the PHA material and resulting materials made during the application of the PHA cake in commercial processes. Small particles will also increase the film formation propensity of PHA particles when they are brought to the melting temperature and begin to flow onto the substrate to which they have been applied.

PHA cake is better suited for developing aqueous PHA (dispersion, emulsion, colloid, suspension, coating) formulations. Keeping the PHA in a wetted state reduces particle agglomeration, and flocculation, resulting in smaller particles that wet more easily, disperse, and otherwise form aqueous formulations. This approach retains the particle size in the PHA cake to less than about fifteen microns in diameter, with about 90% of the particles falling below 8 microns in size. Further, keeping the PHA in cake form without heated drying reduces the cost of manufacture. PHA materials formed using cake will result in minimum film formation temperature reduction as, reduced dewatering rates, increased rheological profiles, and increased barrier performance as compared to those made using PHA having been dried to a powder state.

The PHA cake can be used in a commercial setting to develop aqueous (or any solvent-based) coating materials. Coating describes any PHA-containing dispersion, solution, emulsion, colloid, or suspension. The PHA cake itself can be sold to a customer for future formulation or formulated after manufacture and before sale to a customer. Some end-users of PHA cake include chemical manufacturing companies, paper and substrate manufacturing companies, molded fiber packaging companies, converting companies, and brand owners.

In a further aspect, the present disclosure provides a biodegradable aqueous mixture for coating substrates. According to one embodiment, this aqueous mixture includes from about 35 to about 75 weight percent water and from about 25 to about 65 weight percent solids. The solids in turn are made up of from about 40 to about 99 weight percent polyhydroxyalkanoates based on the total dry weight of the solids. Moreover, the polyhydroxyalkanoates are in the form of polyhydroxyalkanoate particles having a moisture content of no less than about 1% by weight prior to mixing with the water and a Dv (90) particle size of no more than about 10 microns, as determined using ISO 8130-13:2019.

In some embodiments, the polyhydroxyalkanoate particles preferably have a moisture content of at least about 5% by weight prior to mixing with the water

According to certain embodiments, the polyhydroxyalkanoate particles preferably have a Dv (90) particle size of no more than about 8 microns, as determined using ISO 8130-13:2019.

In some instances, polyhydroxyalkanoates have a melting point and the polyhydroxyalkanoate particles are preferably recovered from biomass and subsequent purification processes without the temperature of the polyhydroxyalkanoate particles exceeding a temperature which is about 5° C. below the melting point of the polyhydroxyalkanoates (more preferably about 10° C. below the melting point of the polyhydroxyalkanoates, and even more preferably about 20° C. below the melting point of the polyhydroxyalkanoates), prior to mixing with the water to form the aqueous mixture

According to certain embodiments, the polyhydroxyalkanoate particles are preferably recovered directly from biomass and subsequent purification processes without being dried at a temperature greater than about 95° C., more preferably about 50° C., and even more preferably about 40° C., prior to mixing with the water to form the aqueous mixture.

In some embodiments, the mixture is in the form of a suspension, an emulsion, or a colloid.

In accordance with some embodiments, the solids preferably make up from about 40 to about 50 weight percent polyhydroxyalkanoates based on the total dry weight of the solids.

In some embodiments, the mixture preferably includes from about 45 to about 55 weight percent water and from about 45 to about 55 weight percent solids.

According to certain embodiments, the polyhydroxyalkanoates preferably includes a polyhydroxyalkanoate copolymer made up of from about 75 to about 99 mole percent hydroxybutyrate monomer repeat units and from about 1 to about 25 mole percent monomer repeat units selected from the group consisting of hydroxyvalerate, hydroxyhexanoate, hydroxyoctanoate, and hydroxydecanoate.

In certain embodiments, the polyhydroxyalkanoates preferably includes poly-3-hydroxybutyrate-co-3-hydroxyhexanoate (“P(3HB-co-3HHx)”). More preferably the P(3HB-co-3HHx) is made up of from about 85 to about 98 mole percent hydroxybutyrate and from about 2 to about 15 mole percent hydroxyhexanoate. In certain embodiments, the P(3HB-co-3HHx) is more preferably made up of from about 88 to about 98 mole percent hydroxybutyrate and from about 2 to about 12 mole percent hydroxyhexanoate. In other embodiments, the P(3HB-co-3HHx) is more preferably made up of from about 93 to about 98 mole percent hydroxybutyrate and from about 2 to about 7 mole percent hydroxyhexanoate.

In accordance with some embodiments, the polyhydroxyalkanoates preferably include a polyhydroxyalkanoate terpolymer made up from about 75 to about 99.9 mole percent monomer repeat units of 3-hydroxybutyrate, from about 0.1 to about 25 mole percent monomer repeat units of 3-hydroxyhexanoate, and from about 0.1 to about 25 mole percent monomer repeat units of a third 3-hydoxyalkanoate having from 5 to 12 carbon atoms.

According to certain embodiments, the polyhydroxyalkanoates preferably have a weight average molecular weight from about 50,000 Daltons to about 2.5 million Daltons, as determined by ASTM D5296-05.

In certain embodiments, the polyhydroxyalkanoates more preferably have a weight average molecular weight from about 200,000 Daltons to about 750,000 Daltons, and still more preferably from about 300,000 Daltons to about 550,000 Daltons, as determined by ASTM D5296-05.

In some instances, the solids preferably also include from about 1 weight percent to about 25 weight percent of a polymer selected from the group consisting of poly(lactic acid), poly(caprolactone), poly(ethylene sebecate), poly(butylene succinate), poly(butylene succinate-co-adipate), poly(butylene adipate terephthalate), poly(vinyl acetate), poly(vinyl alcohol), poly(3-hydroxypropionate), polysaccharides, and mixtures thereof.

In some embodiments, the solids more preferably also include poly(lactic acid).

In accordance with some embodiments, the biodegradable aqueous mixture has a Brookfield viscosity from about 1 to about 5,500 centipoise, when measured in accordance with ISO 1652.

In yet another aspect, the present disclosure provides a method for making a coated substrate. According to one embodiment, this method includes a step of providing a paperboard substrate having a first side and a second side. The method also includes a step of applying a layer of an aqueous coating mixture over at least the first side of the substrate.

This coating mixture as applied is made of about 35 to about 75 weight percent water and from about 25 to about 65 weight percent solids. The solids in turn are made up of from about 40 to about 99 weight percent polyhydroxyalkanoates based on the total dry weight of the solids. Moreover, the polyhydroxyalkanoates are in the form of polyhydroxyalkanoate particles having a moisture content of no less than about 1% by weight prior to mixing with the water and a Dv (90) particle size of no more than about 10 microns, as determined using ISO 8130-13:2019.

The method also includes a step of curing the coating mixture to form a continuous coating layer comprising from about 40 to about 99 weight percent polyhydroxyalkanoates.

In certain embodiments, the polyhydroxyalkanoate particles preferably have a moisture content of at least about 5% by weight prior to mixing with the water

According to certain embodiments, the polyhydroxyalkanoate particles preferably have a Dv (90) particle size of no more than about 8 microns, as determined using ISO 8130-13:2019.

In some instances, polyhydroxyalkanoates have a melting point and the polyhydroxyalkanoate particles are preferably recovered from biomass and subsequent purification processes without the temperature of the polyhydroxyalkanoate particles exceeding a temperature which is about 5° C. below the melting point of the polyhydroxyalkanoates (more preferably about 10° C. below the melting point of the polyhydroxyalkanoates, and even more preferably about 20° C. below the melting point of the polyhydroxyalkanoates), prior to mixing with the water to form the aqueous mixture

According to certain embodiments, the polyhydroxyalkanoate particles are preferably recovered directly from biomass and subsequent purification processes without being dried at a temperature greater than about 95° C., more preferably about 50° C., and even more preferably about 40° C., prior to mixing with the water to form the aqueous mixture.

In accordance with some embodiments, the coating mixture preferably is applied in the form of a dispersion, an emulsion, or a colloid.

In accordance with some embodiments, the solids preferably make up from about 40 to about 50 weight percent polyhydroxyalkanoates based on the total dry weight of the solids.

In certain embodiments, coating mixture preferably includes from about 45 to about 55 weight percent water and from about 45 to about 55 weight percent solids.

In some instances, the polyhydroxyalkanoates preferably include a polyhydroxyalkanoate copolymer made up of from about 75 to about 99 mole percent hydroxybutyrate monomer repeat units and from about 1 to about 25 mole percent monomer repeat units selected from the group consisting of hydroxyvalerate, hydroxyhexanoate, hydroxyoctanoate, and hydroxydecanoate.

According to certain embodiments, the polyhydroxyalkanoates preferably includes poly-3-hydroxybutyrate-co-3-hydroxyhexanoate (“P(3HB-co-3HHx)”). More preferably the P(3HB-co-3HHx) is made up of from about 85 to about 98 mole percent hydroxybutyrate and from about 2 to about 15 mole percent hydroxyhexanoate. In certain embodiments, the P(3HB-co-3HHx) is more preferably made up of from about 88 to about 98 mole percent hydroxybutyrate and from about 2 to about 12 mole percent hydroxyhexanoate. In other embodiments, the P(3HB-co-3HHx) is more preferably made up of from about 93 to about 98 mole percent hydroxybutyrate and from about 2 to about 7 mole percent hydroxyhexanoate.

In accordance with some embodiments, the polyhydroxyalkanoates preferably include a polyhydroxyalkanoate terpolymer made up from about 75 to about 99.9 mole percent monomer repeat units of 3-hydroxybutyrate, from about 0.1 to about 25 mole percent monomer repeat units of 3-hydroxyhexanoate, and from about 0.1 to about 25 mole percent monomer repeat units of a third 3-hydoxyalkanoate having from 5 to 12 carbon atoms.

In certain embodiments, the polyhydroxyalkanoates preferably have a weight average molecular weight from about 50,000 Daltons to about 2.5 million Daltons, as determined by ASTM D5296-05.

According to certain embodiments, the polyhydroxyalkanoates more preferably have a weight average molecular weight from about 200,000 Daltons to about 750,000 Daltons, and still more preferably from about 300,000 Daltons to about 550,000 Daltons, as determined by ASTM D5296-05.

In some embodiments, the solids preferably also include from about 1 weight percent to about 25 weight percent of a polymer selected from the group consisting of poly(lactic acid), poly(caprolactone), poly(ethylene sebecate), poly(butylene succinate), poly(butylene succinate-co-adipate), poly(butylene adipate terephthalate), poly(vinyl acetate), poly(vinyl alcohol), poly(3-hydroxypropionate), polysaccharides, and mixtures thereof.

In accordance with some embodiments, the coating mixture more preferably includes poly(lactic acid).

In a further aspect, the present disclosure provides a coated substrate prepared in accordance with the aforementioned method.

In certain embodiments, the coating mixture is preferably applied to the first side of this substrate at a coating weight, after curing, from about 0.5 to about 50 grams per square meter.

In certain embodiments, the paperboard substrate is preferably impregnated with the coating mixture.

### DETAILED DESCRIPTION

The above and other needs are met by a method of forming polyhydroxyalkanoate (PHA) that eliminates heated drying processes, need for re-wetting, increased use of surfactants for dispersing, increased energy usage for high shear dispersing/sonication, significant particle agglomeration resulting in increased particle size as compared to that of those resulting from post lysis. The resulting PHA is newly referred to as PHA cake. The term “cake” is new in this disclosure and has not heretofore been used in the PHA processing industry to describe an end-use material having the properties described above.

PHA cake made according to the methods described here can be either water or solvent-based and is well-suited for formulation of dispersions, colloids, suspensions, coatings, and similar materials. Further, this material is well suited to be used as is into processes which utilize organic and inorganic solvent systems. Examples of formulations made using this cake material include those such as barrier coatings and surface coatings. Further examples for the use of this material include the inclusion or embedding of the electrolytes or material into or onto paper products, dispersions, colloids, emulsions, films, and heat seals. In certain embodiments, the PHA product is a dispersion of PHA in a liquid, such as water or ethanol, but acts much like an emulsion. In other embodiments, the PHA product make be in the form of a suspension, emulsion, or a colloid.

The PHA is created using steps similar to a normal biomass process, but after lysis and protein extraction, the PHA is washed back and forth in an alternating manner with organic and inorganic solvents, such as an alcohol and water, with pressing, terminal filtration, cross flow filtration, decanting, or combination of the two between the washing steps. Blowing air or nitrogen preferably at room temperature can also be performed to yield a PHA cake with no less than about 5% liquid by weight in some embodiments, and in some embodiments, about 60 (+/−10) % PHA and 40 (+/−10) % liquid, by weight. In certain embodiments, the amount of moisture in the PHA cake may be no less than about 1 percent by weight.

The final PHA cake product can then be used in a variety of different ways, such as being placed in a mixer to which water, surfactants, preservatives, rheology modifiers, thickening agents, wetting agents, defoamers, biocides, fillers, binders, and dispersing agents are added. These products can then be used in a variety of different ways, as described elsewhere herein.

By not undergoing a heated drying process with elevated temperatures, the PHA cake retains a smaller PHA particle size, when compared with a standard PHA product that has been baked dry to a solid. The smaller particle size further enables better wetting of the PHA particles, reduction in the time/energy for dispersing of particles, reduction in the use of additives (i.e., surfactants) to prevent flocculation and particle agglomerate, and ultimately increases processability, efficiency, and the sustainability of the PHA material and resulting materials made during the application of the PHA cake in commercial processes. Small particles will also increase the film formation propensity of PHA particles when they are brought to the melting temperature and begin to flow onto the substrate to which they have been applied.

PHA cake is better suited for developing aqueous PHA (dispersion, emulsion, colloid, suspension, coating) formulations. Keeping the PHA in a wetted state reduces particle agglomeration, and flocculation, resulting in smaller particles that wet more easily, disperse, and otherwise form aqueous formulations. This approach retains the particle size in the PHA cake to less than about fifteen microns in diameter, with about 90% of the particles falling below 8 microns in size in some embodiments. In other embodiments, 90% of the particles may be below 10 microns in size. Further, keeping the PHA in cake form without heated drying reduces the cost of manufacture. PHA materials formed using cake will result in minimum film formation temperature reduction as, reduced dewatering rates, increased rheological profiles, and increased barrier performance as compared to those made using PHA having been dried to a powder state.

The PHA cake can be used in a commercial setting to develop aqueous (or any solvent-based) coating materials. Coating describes any PHA-containing dispersion, solution, emulsion, colloid, or suspension. The PHA cake itself can be sold to a customer for future formulation or formulated after manufacture and before sale to a customer. Some end-users of PHA cake include chemical manufacturing companies, paper and substrate manufacturing companies, molded fiber packaging companies, converting companies, and brand owners.

The present disclosure also provides a biodegradable aqueous mixture for coating substrates which incorporates the aforementioned PHA cake.

In general, the aqueous mixture make take the form of either a suspension, an emulsion, or a colloid.

As used in this context, a “suspension” means a heterogeneous mixture of at least two substances, the dispersed material, and the dispersion medium. The particles of a suspension can be separated using filtration. The particles of the suspension will also have a greater propensity to settle under the influence of gravity as compared to certain other mixtures, such as a colloid.

As used herein, a “colloid” is a heterogeneous mixture whose particle size is intermediate between those of a solution and a suspension. The dispersed particles are spread evenly throughout the dispersion medium, i.e., the liquid water. The particles present in a colloid will exhibit a light scattering effect known as the Tyndall effect.

As used herein, an “emulsion” is a type of colloid, wherein an emulsifying agent (such as a surfactant) is present.

At the most general level, the mixture includes water and solids. In general, the mixture comprises from about 35 to about 75 weight percent water and from about 25 to about 65 weight percent solids. In some instances, the mixture more preferably comprises from about 45 to about 55 weight percent water and from about 45 to about 55 weight percent solids.

The solids of the mixture in turn comprise at least polyhydroxyalkanoates and may also comprise other biopolymers and/or additives. Typically, the solids comprise from about 40 to about 99 weight percent polyhydroxyalkanoates, based on the total dry weight of the solids. In some embodiments, the solids more preferably comprise from about 40 to about 50 weight percent polyhydroxyalkanoates, based on the total dry weight of the solids.

The polyhydroxyalkanoates are formed from the aforementioned PHA cake. As discussed above, these polyhydroxyalkanoates are recovered from biomass without the polyhydroxyalkanoates being subjected to extensive heating in order to dry the polyhydroxyalkanoates. Thus, the polyhydroxyalkanoates are in the form of polyhydroxyalkanoate particles having a moisture content of no less than about 1% by weight prior to mixing with the water. In some embodiments, the polyhydroxyalkanoate particles preferably have a moisture content of at least about 5% by weight prior to mixing with the water.

The particles in this PHA cake are observed to have a smaller average particle size as compared to PHA powders which have been fully dried. In particular, the polyhydroxyalkanoate particles preferably have a Dv (90) particle size of no more than about 10 microns, as determined using ISO 8130-13:2019. In some embodiments, the polyhydroxyalkanoate particles preferably have a Dv (90) particle size of no more than about 8 microns, as determined using ISO 8130-13:2019. By comparison, particles of conventional PHA powders which have been fully dried typically have a Dv (90) particle size from about 40 to about 180 microns, as determined using ISO 8130-13:2019.

Without being bound by theory, it is believed that the smaller average size of the polyhydroxyalkanoate particles according to the present disclosure is due to the fact that the polyhydroxyalkanoate particles are not fully dried but rather left with a relatively high moisture content. Consequently, the polyhydroxyalkanoate particles are subjected to milder heating conditions when drying.

In particular, care is taken to ensure that the temperature of the polyhydroxyalkanoate particles is maintained below the melting point of the polyhydroxyalkanoate particles during any drying operations. In general, the polyhydroxyalkanoates have a melting point and the polyhydroxyalkanoate particles are preferably recovered from biomass and subsequent purification processes without the temperature of the polyhydroxyalkanoate particles exceeding a temperature which is about 5° C. below the melting point of the polyhydroxyalkanoates. In certain embodiments, the polyhydroxyalkanoate particles are preferably recovered from biomass and subsequent purification processes without the temperature of the polyhydroxyalkanoate particles exceeding a temperature which is about 10° C. below the melting point of the polyhydroxyalkanoates. In some instances, the polyhydroxyalkanoate particles are even more preferably recovered from biomass and subsequent purification processes without the temperature of the polyhydroxyalkanoate particles exceeding a temperature which is about 20° C. below the melting point of the polyhydroxyalkanoates.

Again, without being bound by theory, it is believed that if the particles are heated to their melting point, then smaller particles of polyhydroxyalkanoate will tend to fuse together to form larger polyhydroxyalkanoate particles. However, if the temperature is maintained well below the melting point of the polyhydroxyalkanoates, then the polyhydroxyalkanoate particles will tend to retain a smaller average particle size.

There are a variety of forms of polyhydroxyalkanoate, and it will be appreciated that the exact melting point of the polyhydroxyalkanoate particles will depend on which forms of polyhydroxyalkanoate are present in the particles.

In general, the polyhydroxyalkanoate particles are typically recovered directly from biomass and subsequent purification processes without being dried at a temperature greater than about 95° C. In certain embodiments, the polyhydroxyalkanoate particles are preferably recovered directly from biomass and subsequent purification processes without being dried at a temperature greater than about 50° C. Even more preferably, the polyhydroxyalkanoate particles are recovered directly from biomass and subsequent purification processes without being dried at a temperature greater than about 40° C.

Again, a variety of forms of polyhydroxyalkanoate may be used in the PHA cake and in the aqueous mixture formed therefrom. In some instances, the polyhydroxyalkanoate may be a homopolymer, such as polyhydroxybutyrate. More typically, the polyhydroxyalkanoate is a copolymer or a terpolymer.

For instance, in certain embodiments, the polyhydroxyalkanoates preferably comprise a polyhydroxyalkanoate copolymer made up of from about 75 to about 99 mole percent hydroxybutyrate monomer repeat units and from about 1 to about 25 mole percent monomer repeat units selected from the group consisting of hydroxyvalerate, hydroxyhexanoate, hydroxyoctanoate, and hydroxydecanoate.

As an example, in certain embodiments, the polyhydroxyalkanoates preferably comprise poly-3-hydroxybutyrate-co-3-hydroxyhexanoate (“P(3HB-co-3HHx)”). More preferably this P(3HB-co-3HHx) comprises from about 85 to about 98 mole percent hydroxybutyrate and from about 2 to about 15 mole percent hydroxyhexanoate. In certain embodiments, the P(3HB-co-3HHx) more preferably comprises from about 88 to about 98 mole percent hydroxybutyrate and from about 2 to about 12 mole percent hydroxyhexanoate. In other embodiments, the P(3HB-co-3HHx) more preferably comprises from about 93 to about 98 mole percent hydroxybutyrate and from about 2 to about 7 mole percent hydroxyhexanoate.

In other embodiments, the polyhydroxyalkanoates preferably comprise a polyhydroxyalkanoate terpolymer made up of from about 75 to about 99.9 mole percent monomer repeat units of 3-hydroxybutyrate, from about 0.1 to about 25 mole percent monomer repeat units of 3-hydroxyhexanoate, and from about 0.1 to about 25 mole percent monomer repeat units of a third 3-hydoxyalkanoate having from 5 to 12 carbon atoms.

In terms of molecular weight, the polyhydroxyalkanoates preferably have a weight average molecular weight from about 50,000 Daltons to about 2.5 million Daltons, as determined by ASTM D5296-05. More preferably, the polyhydroxyalkanoates have a weight average molecular weight from about 200,000 Daltons to about 750,000 Daltons, and still more preferably from about 300,000 Daltons to about 550,000 Daltons, as determined by ASTM D5296-05.

As noted above, the solids of the aqueous mixture may also include other biopolymers. In some instances, the solids preferably comprise from about 1 weight percent to about 25 weight percent of a polymer selected from the group consisting of poly(lactic acid), poly(caprolactone), poly(ethylene sebecate), poly(butylene succinate), poly(butylene succinate-co-adipate), poly(butylene adipate terephthalate), poly(vinyl acetate), poly(vinyl alcohol), poly(3-hydroxypropionate), polysaccharides, and mixtures thereof. In some instances, it is particularly preferred that the solids comprise of poly(lactic acid).

In addition, the aqueous mixture may include various additives such as preservatives, rheology modifiers, plasticizers, fillers, nucleating agents, dispersing, and wetting agents in order to improve the stability of the aqueous mixture and/or to improve the material properties of the coated layer formed from the mixture. Surprisingly, however, it has been observed that the aqueous mixture using the PHA cake according to the present disclosure, may be prepared using a smaller amount of additives, as compared to PHA mixtures prepared using PHA powders which are fully dried prior to mixing.

Once prepared, the biodegradable aqueous mixture typically has a Brookfield viscosity from about 1 to about 5,500 centipoise, when measured in accordance with ISO 1652. More preferably, the biodegradable aqueous mixture has a Brookfield viscosity from about 100 to about 1200 centipoise, when measured in accordance with ISO 1652. In certain embodiments, the biodegradable aqueous mixture even more preferably has a Brookfield viscosity from about 100 to about 500 centipoise, when measured in accordance with ISO 1652.

The present disclosure also provides a method for making a coated substrate, using the aforementioned aqueous mixture as a coating mixture, and a coated substrate prepared according to this method. According to the method, a paperboard substrate is provided having both a first side and a second side. A layer of aqueous coating mixture is then applied over at least the first side of the substrate. In some instances, the coating mixture may be applied over both the first side and the second side of the substrate.

Finally, the coating mixture is cured to form a continuous coating layer comprising polyhydroxyalkanoates. Curing of the coating layer is preferably carried out by heating the substrate and coating to a temperature of about 105° C. to 145° C. in order to evaporate the water from the coating mixture and fuse the solids particles together into a continuous layer.

The coating mixture is as discussed above. Thus, the coating mixture as applied generally comprises from about 35 to about 75 weight percent water and from about 25 to about 65 weight percent solids, more preferably from about 45 to about 55 weight percent water and from about 45 to about 55 weight percent solids.

The solids comprise from about 40 to about 99 weight percent polyhydroxyalkanoates based on the total dry weight of the solids, more preferably from about 40 to about 50 weight percent polyhydroxyalkanoates based on the total dry weight of the solids.

Moreover, the polyhydroxyalkanoates are in the form of polyhydroxyalkanoate particles having a moisture content of no less than about 1% (more preferably at least about 5%) by weight prior to mixing with the water and a Dv (90) particle size of no more than about 10 microns (more preferably about 8 microns), as determined using ISO 8130-13:2019.

As for the finished coated substrate, the coating mixture is preferably applied to the first side of this substrate at a coating weight, after curing, from about 0.5 to about 50 grams per square meter.

As noted above, a coating layer may in some instances be applied to both the first side and the second side of the substrate.

Moreover, in some instance the paperboard substrate may be fully impregnated with the coating mixture. This may be achieved by metered size pressing of the paperboard substrates, for example.

### EMBODIMENTS

The present disclosure is also further illustrated by the following embodiments:

Embodiment 1. A biodegradable aqueous mixture for coating substrates, the mixture comprising:

from about 35 to about 75 weight percent water and from about 25 to about 65 weight percent solids,

wherein the solids comprise from about 40 to about 99 weight percent polyhydroxyalkanoates based on the total dry weight of the solids, and

wherein the polyhydroxyalkanoates are in the form of polyhydroxyalkanoate particles having a moisture content of at least about 1% by weight prior to mixing with the water and a Dv (90) particle size of no more than about 10 microns, as determined using ISO 8130-13:2019.

Embodiment 2. The biodegradable aqueous mixture of Embodiment 1, wherein the polyhydroxyalkanoate particles have a moisture content of at least about 5% by weight prior to mixing with the water

Embodiment 3. The biodegradable aqueous mixture of Embodiments 1 or 2, wherein the polyhydroxyalkanoate particles have a Dv (90) particle size of no more than about 10 microns, as determined using ISO 8130-13:2019.

Embodiment 4. The biodegradable aqueous mixture of any of the preceding embodiments, wherein the polyhydroxyalkanoates have a melting point and the polyhydroxyalkanoate particles are recovered from biomass and subsequent purification processes without the temperature of the polyhydroxyalkanoate particles exceeding a temperature which is about 5° C. below the melting point of the polyhydroxyalkanoates, and more preferably about 10° C. below the melting point of the polyhydroxyalkanoates, prior to mixing with the water to form the aqueous mixture

Embodiment 5. The biodegradable aqueous mixture of any of the preceding embodiments, wherein the polyhydroxyalkanoate particles are recovered from biomass and subsequent purification processes without the temperature of the polyhydroxyalkanoate particles exceeding about 95° C., preferably about 50° C., and more preferably about 40° C., prior to mixing with the water to form the aqueous mixture.

Embodiment 6. The biodegradable aqueous mixture of any of the preceding embodiments, wherein the mixture is in the form of a suspension, an emulsion, or a colloid.

Embodiment 7. The biodegradable aqueous mixture of any of the preceding embodiments, wherein the solids comprise from about 40 to about 50 weight percent polyhydroxyalkanoates based on the total dry weight of the solids.

Embodiment 8. The biodegradable aqueous mixture of any of the preceding embodiments, wherein the mixture comprises from about 45 to about 55 weight percent water and from about 45 to about 55 weight percent solids.

Embodiment 9. The biodegradable aqueous mixture of any of the preceding embodiments, wherein the polyhydroxyalkanoates comprise a polyhydroxyalkanoate copolymer comprising from about 75 to about 99 mole percent hydroxybutyrate monomer repeat units and from about 1 to about 25 mole percent monomer repeat units selected from the group consisting of hydroxyvalerate, hydroxyhexanoate, hydroxyoctanoate, and hydroxydecanoate.

Embodiment 10. The biodegradable aqueous mixture of any of the preceding embodiments, wherein the polyhydroxyalkanoates comprise poly-3-hydroxybutyrate-co-3-hydroxyhexanoate (“P(3HB-co-3HHx)”).

Embodiment 11. The biodegradable aqueous mixture of Embodiment 10, wherein the P(3HB-co-3HHx) comprises from about 85 to about 98 mole percent hydroxybutyrate and from about 2 to about 15 mole percent hydroxyhexanoate, more preferably from about 88 to about 98 mole percent hydroxybutyrate and from about 2 to about 12 mole percent hydroxyhexanoate, and still more preferably from about 93 to about 98 mole percent hydroxybutyrate and from about 2 to about 7 mole percent hydroxyhexanoate.

Embodiment 12. The biodegradable aqueous mixture of any of Embodiments 1-8, wherein the polyhydroxyalkanoates comprise a polyhydroxyalkanoate terpolymer made up from about 75 to about 99.9 mole percent monomer repeat units of 3-hydroxybutyrate, from about 0.1 to about 25 mole percent monomer repeat units of 3-hydroxyhexanoate, and from about 0.1 to about 25 mole percent monomer repeat units of a third 3-hydroxyalkanoate having from 5 to 12 carbon atoms.

Embodiment 13. The biodegradable aqueous mixture of any of the preceding embodiments, wherein the polyhydroxyalkanoates have a weight average molecular weight from about 50,000 Daltons to about 2.5 million Daltons, and more preferably from about 200,000 Daltons to about 750,000 Daltons, and still more preferably from about 300,000 Daltons to about 550,000 Daltons, as determined by ASTM D5296-05.

Embodiment 14. The biodegradable aqueous mixture of any of the preceding embodiments, wherein the solids further comprise from about 1 weight percent to about 25 weight percent, based on the total dry weight of the solids, of a polymer selected from the group consisting of poly(lactic acid), poly(caprolactone), poly(ethylene sebecate), poly(butylene succinate), poly(butylene succinate-co-adipate), poly(butylene adipate terephthalate), poly(vinyl acetate), poly(vinyl alcohol), poly(3-hydroxypropionate), polysaccharides and mixtures thereof.

Embodiment 15. The biodegradable aqueous mixture of any of the preceding embodiments, wherein the solids further comprise poly(lactic acid).

Embodiment 16. The biodegradable aqueous mixture of any of the preceding embodiments, wherein the biodegradable aqueous mixture has a Brookfield viscosity from about 1 to about 5,500 centipoise, when measured in accordance with ISO 1652.

Embodiment 17. A method for making a coated substrate, comprising the steps of:

providing a paperboard substrate having a first side and a second side;

applying a layer of an aqueous coating mixture over at least the first side of the substrate,

wherein the coating mixture as applied comprises from about 35 to about 75 weight percent water and from about 25 to about 65 weight percent solids,

wherein the solids comprise from about 40 to about 99 weight percent polyhydroxyalkanoates based on the total dry weight of the solids, and

wherein the polyhydroxyalkanoates are in the form of polyhydroxyalkanoate particles having a moisture content of at least about 1% by weight prior to mixing with the water and a Dv (90) particle size of no more than about 10 microns, as determined using ISO 8130-13:2019; and

curing the coating mixture to form a continuous coating layer comprising from about 40 to about 99 weight percent polyhydroxyalkanoates.

Embodiment 18. The method of Embodiment 17, wherein the polyhydroxyalkanoate particles have a moisture content of at least about 5% by weight prior to mixing with the water

Embodiment 19. The method of Embodiments 17 or 18, wherein the polyhydroxyalkanoate particles have a Dv (90) particle size of no more than about 8 microns, as determined using ISO 8130-13:2019.

Embodiment 20. The method of any of Embodiments 17-19, wherein the polyhydroxyalkanoates have a melting point and the polyhydroxyalkanoate particles are recovered from biomass and subsequent purification processes without the temperature of the polyhydroxyalkanoate particles exceeding a temperature which is about 5° C. below the melting point of the polyhydroxyalkanoates, and more preferably about 10° C. below the melting point of the polyhydroxyalkanoates. ° C., prior to mixing with the water to form the aqueous mixture.

Embodiment 21. The method of any of Embodiments 17-20, wherein the polyhydroxyalkanoate particles are recovered from biomass and subsequent purification processes without the temperature of the polyhydroxyalkanoate particles exceeding about 95° C., preferably about 50° C., and more preferably about 40° C., prior to mixing with the water to form the aqueous mixture.

Embodiment 22. The method of any of Embodiments 17-21, wherein the coating mixture is applied in the form of a suspension, an emulsion, or a colloid.

Embodiment 23. The method of any of Embodiments 17-22, wherein the solids comprise from about 40 to about 50 weight percent polyhydroxyalkanoates based on the total dry weight of the solids.

Embodiment 24. The method of any of Embodiments 17-23, wherein the coating mixture comprises from about 45 to about 55 weight percent water and from about 45 to about 55 weight percent solids.

Embodiment 25. The method of any of Embodiments 17-24, wherein the polyhydroxyalkanoates comprise a polyhydroxyalkanoate copolymer comprising from about 75 to about 99 mole percent hydroxybutyrate monomer repeat units and from about 1 to about 25 mole percent monomer repeat units selected from the group consisting of hydroxyvalerate, hydroxyhexanoate, hydroxyoctanoate, and hydroxydecanoate.

Embodiment 26. The method of any of Embodiments 17-25, wherein the polyhydroxyalkanoates comprise poly-3-hydroxybutyrate-co-3-hydroxyhexanoate (“P(3HB-co-3HHx)”).

Embodiment 27. The method of Embodiment 26, wherein the P(3HB-co-3HHx) comprises from about 85 to about 98 mole percent hydroxybutyrate and from about 2 to about 15 mole percent hydroxyhexanoate, more preferably from about 88 to about 98 mole percent hydroxybutyrate and from about 2 to about 12 mole percent hydroxyhexanoate, and still more preferably from about 93 to about 98 mole percent hydroxybutyrate and from about 2 to about 7 mole percent hydroxyhexanoate

Embodiment 28. The method of any of Embodiments 17-24, wherein the polyhydroxyalkanoates comprise a polyhydroxyalkanoate terpolymer made up from about 75 to about 99.9 mole percent monomer repeat units of 3-hydroxybutyrate, from about 0.1 to about 25 mole percent monomer repeat units of 3-hydroxyhexanoate, and from about 0.1 to about 25 mole percent monomer repeat units of a third 3-hydroxyalkanoate having from 5 to 12 carbon atoms.

Embodiment 29. The method of any of Embodiments 17-28, wherein the polyhydroxyalkanoates have a weight average molecular weight from about 50,000 Daltons to about 2.5 million Daltons, and more preferably from about 200,000 Daltons to about 750,000 Daltons, and still more preferably from about 300,000 Daltons to about 550,000 Daltons, as determined by ASTM D5296-05.

Embodiment 30. The method of any of Embodiments 17-29, wherein the solids further comprise from about 1 weight percent to about 25 weight percent, based on the total dry weight of the solids, of a polymer selected from the group consisting of poly(lactic acid), poly(caprolactone), poly(ethylene sebecate), poly(butylene succinate), poly(butylene succinate-co-adipate), poly(butylene adipate terephthalate), poly(vinyl acetate), poly(vinyl alcohol), poly(3-hydroxypropionate), polysaccharides and mixtures thereof.

Embodiment 31. The method of any of Embodiments 17-30, wherein the solids further comprise poly(lactic acid).

Embodiment 32. A coated substrate prepared according to the method of Embodiment 17.

Embodiment 33. The coated substate of Embodiment 32, wherein the coating mixture is applied the first side of the substrate at a coating weight, after curing, from about 0.5 to about 50 grams per square meter.

Embodiment 34. The coated substrate of Embodiments 32 or 33, wherein the paperboard substrate is impregnated with the coating mixture.

Embodiment 35. Polyhydroxyalkanoate (PHA) cake that is formed directly from biomass and subsequent purification processes absent any heated drying step, with a moisture content of no less than about 5% by weight, and a Dv (90) particle size of no more than about 8 microns.

Embodiment 36. PHA cake that is formed directly from biomass and subsequent purification processes absent any heated drying step, with a moisture content of no less than about 5% by weight, and a Dv (90) particle size greater than about 8 microns.

Embodiment 37. The PHA cake of Embodiments 35 or 36, where as they are combined as a mixture of particle sizes or matrix of particles to provide liquid barriers where temporary clogging is needed.

Embodiment 38. The PHA cake of Embodiment 35, comprising homopolymer, copolymer, block copolymer, branched copolymer, and terpolymer PHAs or combinations thereof.

Embodiment 39. The PHA cake of Embodiment 35, comprising at least one of short-chain, medium-chain, and long-chain PHAs including at least one of such as butyrate, propionate, valerate, hexanoate, octanoate, and decanoate.

Embodiment 40. The PHA cake of Embodiment 35, PHA cake comprising a final dry mass content of from about 30% to about 95% by weight.

Embodiment 41. The PHA cake of Embodiment 35, wherein the PHA is formed in a biological process.

Embodiment 42. The PHA cake of Embodiment 35, wherein the PHA is formed by fermentation.

Embodiment 43. The PHA cake of Embodiment 35, wherein the PHA is formed via reactive synthesis.

Embodiment 44. The PHA cake of Embodiment 35, comprising PHA content of from about 30% to about 95% by weight.

Embodiment 45. The PHA cake of Embodiment 35, wherein the PHA is produced from a combination of biological and non-biological processes.

Embodiment 46. The PHA cake of Embodiment 35, wherein the PHA cake is separated and purified from production precursors and then filtered through at least one of mechanical dewatering and mechanical desolventing systems.

Embodiment 47. The PHA cake of Embodiment 35, wherein the PHA cake is mechanically dewatered using at least one of organic solvents, inorganic solvents, and solvent-free systems.

Embodiment 48. The PHA cake of Embodiment 35, wherein the PHA cake is mechanically dewatered using a multi-step process.

Embodiment 49. The PHA cake of Embodiment 35, wherein the PHA cake is used in a non-extrusion-based process or application.

Embodiment 50. The PHA cake of Embodiment 35, wherein the PHA cake is used in at least one of an aqueous coating, a solvent coating, emulsion, dispersion, colloid, electrolyte, and suspension.

Embodiment 51. The PHA cake of Embodiment 35, wherein the PHA cake is used in at least one of an aqueous coating, a solvent coating, a dispersion, a colloid, a suspension, and an emulsion, which is used for at least one of inks, barrier coatings, surface coatings, embedded coatings, paper products, films, heat seals, cosmetics, personal care, home care, water treatment, filtration, media, water resistant coatings, and oil resistant coatings.

Embodiment 52. Polyhydroxyalkanoate (PHA) cake formed by:

biological production of PHA,

lysis,

cellular debris removal,

purification of the PHA with alternating washings using organic and inorganic liquids,

pressing/decanting of the PHA between the washings, and

partial drying of the PHA with room temperature gas and without any heated drying step,

thereby producing the PHA cake with a moisture content of no less than about 5% by weight, and a tuned application-specific particle size.

As used herein, the phrase “at least one of A, B, and C” means all possible combinations of none or multiple instances of each of A, B, and C, but at least one A, or one B, or one C. For example, and without limitation: Ax1, Ax2+Bx1, Cx2, Ax1+Bx1+Cx1, Ax7+Bx12+Cx113. It does not mean Ax0+Bx0+Cx0.

The foregoing description of embodiments for this invention has been presented for purposes of illustration and description. It is not intended to be exhaustive or to limit the invention to the precise form disclosed. Obvious modifications or variations are possible in light of the above teachings. The embodiments are chosen and described in an effort to provide illustrations of the principles of the invention and its practical application, and to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

What is claimed is:

1. A biodegradable aqueous mixture for coating substrates, the mixture comprising:
from about 35 to about 75 weight percent water and from about 25 to about 65 weight percent solids,
wherein the solids comprise from about 40 to about 99 weight percent polyhydroxyalkanoates based on the total dry weight of the solids, and
wherein the polyhydroxyalkanoates are in the form of polyhydroxyalkanoate particles having a moisture content of at least about 1% by weight prior to mixing with the water and a Dv (90) particle size of no more than about 10 microns, as determined using ISO 8130-13:2019.

2. The biodegradable aqueous mixture of claim 1, wherein the polyhydroxyalkanoate particles have a moisture content of at least about 5% by weight prior to mixing with the water.

3. The biodegradable aqueous mixture of claim 1, wherein the polyhydroxyalkanoate particles have a Dv (90) particle size of no more than about 8 microns, as determined using ISO 8130-13:2019.

4. The biodegradable aqueous mixture of claim 1, wherein the polyhydroxyalkanoates have a melting point and the polyhydroxyalkanoate particles are recovered from biomass and subsequent purification processes without the temperature of the polyhydroxyalkanoate particles exceeding a temperature which is about 5° C. below the melting point of the polyhydroxyalkanoates, prior to mixing with the water to form the aqueous mixture.

5. The biodegradable aqueous mixture of claim 1, wherein the polyhydroxyalkanoate particles are recovered from biomass and subsequent purification processes without the temperature of the polyhydroxyalkanoate particles exceeding about 95° C., prior to mixing with the water to form the aqueous mixture.

6. The biodegradable aqueous mixture of claim 1, wherein the mixture is in the form of a suspension, an emulsion, or a colloid.

7. The biodegradable aqueous mixture of claim 1, wherein the solids comprise from about 40 to about 50 weight percent polyhydroxyalkanoates based on the total dry weight of the solids.

8. The biodegradable aqueous mixture of claim 1, wherein the mixture comprises from about 45 to about 55 weight percent water and from about 45 to about 55 weight percent solids.

9. The biodegradable aqueous mixture of claim 1, wherein the polyhydroxyalkanoates comprise a polyhydroxyalkanoate copolymer comprising from about 75 to about 99 mole percent hydroxybutyrate monomer repeat units and from about 1 to about 25 mole percent monomer repeat units selected from the group consisting of hydroxyvalerate, hydroxyhexanoate, hydroxyoctanoate, and hydroxydecanoate.

10. The biodegradable aqueous mixture of claim 1, wherein the polyhydroxyalkanoates comprise poly-3-hydroxybutyrate-co-3-hydroxyhexanoate (“P(3HB-co-3HHx)”).

11. The biodegradable aqueous mixture of claim 10, wherein the P(3HB-co-3HHx) comprises from about 85 to about 98 mole percent hydroxybutyrate and from about 2 to about 15 mole percent hydroxyhexanoate.

12. The biodegradable aqueous mixture of claim 1, wherein the polyhydroxyalkanoates comprise a polyhydroxyalkanoate terpolymer made up from about 75 to about 99.9 mole percent monomer repeat units of 3-hydroxybutyrate, from about 0.1 to about 25 mole percent monomer repeat units of 3-hydroxyhexanoate, and from about 0.1 to about 25 mole percent monomer repeat units of a third 3-hydroxyalkanoate having from 5 to 12 carbon atoms.

13. The biodegradable aqueous mixture of claim 1, wherein the polyhydroxyalkanoates have a weight average molecular weight from about 50,000 Daltons to about 2.5 million Daltons, as determined by ASTM D5296-05.

14. The biodegradable aqueous mixture of claim 1, wherein the solids further comprise from about 1 weight percent to about 25 weight percent, based on the total dry weight of the solids, of a polymer selected from the group consisting of poly(lactic acid), poly(caprolactone), poly(ethylene sebecate), poly(butylene succinate), poly(butylene succinate-co-adipate), poly(butylene adipate terephthalate), poly(vinyl acetate), poly(vinyl alcohol), poly(3-hydroxypropionate), polysaccharides, and mixtures thereof.

15. The biodegradable aqueous mixture of claim 1, wherein the solids further comprise poly(lactic acid).

16. The biodegradable aqueous mixture of claim 1, wherein the biodegradable aqueous mixture has a Brookfield viscosity from about 1 to about 5,500 centipoise, when measured in accordance with ISO 1652.

17. A method for making a coated substrate, comprising the steps of:
providing a paperboard substrate having a first side and a second side;
applying a layer of an aqueous coating mixture over at least the first side of the substrate,
wherein the coating mixture as applied comprises from about 35 to about 75 weight percent water and from about 25 to about 65 weight percent solids,
wherein the solids comprise from about 40 to about 99 weight percent polyhydroxyalkanoates based on the total dry weight of the solids, and
wherein the polyhydroxyalkanoates are in the form of polyhydroxyalkanoate particles having a moisture content of at least about 1% by weight prior to mixing with the water and a Dv (90) particle size of no more than about 10 microns, as determined using ISO 8130-13:2019; and
curing the coating mixture to form a continuous coating layer comprising from about 40 to about 99 weight percent polyhydroxyalkanoates.

18. The method of claim 17, wherein the polyhydroxyalkanoate particles have a moisture content of at least about 5% by weight prior to mixing with the water

19. The method of claim 17, wherein the polyhydroxyalkanoate particles have a Dv (90) particle size of no more than about 8 microns, as determined using ISO 8130-13:2019.

20. The method of claim 17, wherein the polyhydroxyalkanoates have a melting point and the polyhydroxyalkanoate particles are recovered from biomass and subsequent purification processes without the temperature of the polyhydroxyalkanoate particles exceeding a temperature which is about 5° C. below the melting point of the polyhydroxyalkanoates, prior to mixing with the water to form the coating mixture.

21. The method of claim 17, wherein the polyhydroxyalkanoate particles are recovered from biomass and subsequent purification processes without the temperature of the polyhydroxyalkanoate particles exceeding about 95° C., prior to mixing with the water to form the coating mixture.

22. The method of claim 17, wherein the coating mixture is applied in the form of a suspension, an emulsion, or a colloid.

23. The method of claim 17, wherein the solids comprise from about 40 to about 50 weight percent polyhydroxyalkanoates based on the total dry weight of the solids.

24. The method of claim 17, wherein the coating mixture comprises from about 45 to about 55 weight percent water and from about 45 to about 55 weight percent solids.

25. The method of claim 17, wherein the polyhydroxyalkanoates comprise a polyhydroxyalkanoate copolymer comprising from about 75 to about 99 mole percent hydroxybutyrate monomer repeat units and from about 1 to about 25 mole percent monomer repeat units selected from the group consisting of hydroxyvalerate, hydroxyhexanoate, hydroxyoctanoate, and hydroxydecanoate.

26. The method of claim 17, wherein the polyhydroxyalkanoates comprise poly-3-hydroxybutyrate-co-3-hydroxyhexanoate (“P(3HB-co-3HHx)”).

27. The method of claim 26, wherein the P(3HB-co-3HHx) comprises from about 85 to about 98 mole percent hydroxybutyrate and from about 2 to about 15 mole percent hydroxyhexanoate.

28. The method of claim 17, wherein the polyhydroxyalkanoates comprise a polyhydroxyalkanoate terpolymer made up from about 75 to about 99.9 mole percent monomer repeat units of 3-hydroxybutyrate, from about 0.1 to about 25 mole percent monomer repeat units of 3-hydroxyhexanoate, and from about 0.1 to about 25 mole percent monomer repeat units of a third 3-hydroxyalkanoate having from 5 to 12 carbon atoms.

29. The method of claim 17 wherein the polyhydroxyalkanoates have a weight average molecular weight from about 50,000 Daltons to about 2.5 million Daltons, as determined by ASTM D5296-05.

30. The method of claim 17, wherein the solids further comprise from about 1 weight percent to about 25 weight percent, based on the total dry weight of the solids, of a polymer selected from the group consisting of poly(lactic acid), poly(caprolactone), poly(ethylene sebecate), poly(butylene succinate), poly(butylene succinate-co-adipate), poly(butylene adipate terephthalate), poly(vinyl acetate), poly(vinyl alcohol), poly(3-hydroxypropionate), polysaccharides and mixtures thereof.

31. The method of claim 17, wherein the solids further comprise poly(lactic acid).

32. A coated substrate prepared according to the method of claim 17.

33. The coated substate of claim 32, wherein the coating mixture is applied the first side of the substrate at a coating weight, after curing, from about 0.5 to about 50 grams per square meter.

34. The coated substrate of claim 32, wherein the paperboard substrate is impregnated with the coating mixture.
